(19) United States
(12) Patent Application Publication
(10) Pub. No.: US 2018/0280542 A1
(43) Pub. Date: Oct. 4, 2018

(54) PORTABLE BIOLUMINESCENCE SYSTEMS AND METHODS FOR IN VIVO MONITORING OF MOLECULAR AND METABOLIC EVENTS IN ANIMALS

(71) Applicant: Ecole Polytechnique Fédérale de Lausanne (EPFL), Lausanne (CH)

(72) Inventors: Elena A. Goun, Saint-Sulpice (CH); Aurélien Godinat, Ecublens (CH); Aleksey Yevtodiyenko, Renens (CH)

(21) Appl. No.: 15/944,714

(22) Filed: Apr. 3, 2018

Related U.S. Application Data
(60) Provisional application No. 62/481,395, filed on Apr. 4, 2017.

(51) Int. Cl. A61K 49/00; A61B 5/00; C12Q 1/66; C07D 277/64

(52) U.S. Cl. CPC A61K 49/0013; C07D 277/64; C12Q 1/66; A61B 5/0059

(57) ABSTRACT

A system for monitoring biological processes in vivo is provided. The system comprises an implantable luciferase biosensor comprising luciferase in a biocompatible matrix and a caged luciferin probe. The caged luciferin probe can be administered to a living subject and upon encountering a biological activity, e.g., enzyme activity, the caged luciferin probe releases free luciferin which can then interact with the biosensor luciferase to generate light. The light can then be detected and/or measured by a light detector. Compositions, methods and kits related to the system are provided herein.

### CROSS-REFERENCE TO RELATED APPLICATIONS

This application claims the benefit of U.S. Provisional Application No. 62/481,395 filed Apr. 4, 2017, incorporated by reference herein.

### TECHNICAL FIELD

This disclosure relates to bioluminescence-based monitoring of molecular and metabolic events in living animals. Portable bioluminescence systems for in vivo monitoring of biological processes is described. The system comprises an implantable luciferase based sensor, caged luciferin probes, and a portable highly sensitive light detector. Methods for use of these bioluminescence systems are also described. The systems and methods are applicable for monitoring molecular and metabolic events in living animals and potentially in humans in a variety of normal and medical conditions.

### BACKGROUND

In vivo bioluminescent imaging (BLI) is a rapidly growing optical imaging modality widely used for preclinical research and drug development in a range of therapeutic areas, including cancer, infectious diseases and stem cell research. BLI has been successfully used to investigate gene expression, protein-protein interactions, enzyme activity and cell tracking (Contag et al., 1995, Mol Microbiol, 18:593-603; Sadikot and Blackwell, 2008, Methods Mol Biol, 477:383-394; de Almeida et al., 2011, Am J Physiol Heart Circ Physiol, 301:H663-671; Badr and Tannous, 2011, Trends Biotechnol, 29:624-633).

BLI relies on the use of luciferase enzyme that generates photon emission as a result of interaction with its substrate luciferin. Several types of naturally occurring luciferase/luciferin systems were isolated from non-mammalian species such as green or red click beetle luciferase/D-luciferin, Renilla luciferase/Coelenterazine, Gaussia luciferase/Coelenterazine, bacterial luciferase/Aliphatic aldehyde and reduced flavin mononucleotide phosphate. The firefly luciferase (Fluc)/D-luciferin pair is currently the most commonly exploited BLI reporter system due to convenient in vivo kinetics and red-shifted light emission (530-640 nm) with improved tissue penetration properties. Fluc catalyzes the oxidation of D-luciferin in the presence of ATP and magnesium as cofactors (FIG. 1A).

A prototypical BLI study involves injection of luciferase-transfected cells into mice or rats then imaging cell growth and metastasis in the presence of saturating doses of luciferin. More recently, “caged” luciferin based probes were developed that open the possibility to use luciferase-expressing cells as a biosensor/reporter for a specific metabolic and molecular assays to sense molecular signatures of a large variety of specific enzymes, metabolites or pharmaceutical compounds (Vorobyeva et al., 2015, 10:e0131037; Godinat et al., 2013, ACS Chem Biol, 8:987-999; Godinat et al., 2014, Curr Protoc Chem Biol, 6:169-189). These BLI probes rely on the principle that chemically modified luciferin, e.g., a luciferin conjugate, is not a substrate for luciferase until it gets released or “uncaged” by a specific biological process of interest (e.g., enzyme cleavage) (FIGS. 1B and 2). Consequently, intensity of bioluminescence quantitatively correlates with the amount of free luciferin in animals that in turn reflects activity of targeted enzyme or concentration of selected metabolite. A variety of luciferin-based probes have been designed and patented. Examples of caged luciferin probes include peroxy-caged luciferin for monitoring oxidative stress (see, e.g., U.S. Pat. Appn. Pub. Nos. 2013/0315829 and 2015/0376680, U.S. Pat. No. 8,937,183, and PCT Pub. No. WO 2011/133800 A1); caspase substrate-caged luciferin for detection apoptosis and apoptosis-related diseases (see, e.g., U.S. Pat. No. 7,148,030, U.S. Pat. Appn. Pub. Nos. 2010/0303728 and 2009/0238766); complementary caged luciferin precursors for simultaneous monitoring oxidative stress and inflammatory processes (see, e.g., U.S. Pat. No. 9,173,966 and U.S. Pat. Appn. Pub. No. 2013/0287699 A1; organophosphine-caged luciferin for detection fluxes of low molecular weight (MW) metabolites, including glucose and lactate (see, e.g., PCT Pub. No. WO 2014/111906 A1); fatty acid-caged luciferin for detection of adipose tissue, fatty acid uptake rate, etc. (see, e.g., U.S. Pat. Appn. Pub. No. 2014/0199239); beta-lactam caged luciferin for evaluation of pathogen resistance to beta-lactam antibiotics (see, e.g., U.S. Pat. Appn. Pub. No. 2009/0246862); and galactoside-caged luciferin for investigation beta-galactosidase activity as well as cell to cell interaction (see, e.g., U.S. Pat. No. 7,582,417).

Several sophisticated and highly sensitive stationary BLI instruments have been developed and are commercially available for investigating cellular and molecular events in living animals. Currently used stationary BLI instruments include IVIS Spectrum (Perkin Elmer), NightOWL II (Berthold Technologies), Photometrics high-performance CMOS, EMCCD and CCD and LI-COR Biosciences (Pearl Trilogy Imager) cameras. These instruments are currently used for all BLI studies performed on small animals such as mice and rats.

Currently, stationary BLI instruments are typically used for in vivo measurement of luminescence generated in the presence of uncaged luciferin administered to animals having or engineered to have luciferase-expressing cells. Despite great sensitivity and increasing number of applications of the current BLI methods, the use of the luciferase-expressing cells as a sensor/reporter with caged luciferin probes and detection by stationary BLI instruments, has some significant disadvantages. Transplanted luciferase-expressing cells may proliferate and accidentally leak from the sensor. This restricts a potential use of this type of the sensor in humans because of the risk of spreading of the transplanted cells. The metabolic state of the transplanted cells may change over time, which in turn may lead to variations in the level of luciferase expression. Thus the signal, e.g., light emission, from this sensor will depend not only from the level of the uncaged luciferin but also on amount of available luciferase. Currently, in vivo BLI is limited to the use the currently available transgenic mice that express luciferase enzyme either ubiquitously or in specific organs or in rodents transplanted with luciferase-expressing cells. Unfortunately, these represent only a small percentage of existing animal models of human disease. The current technologies for detecting and measuring a bioluminescent signal in vivo are also limited to the use in rodents by available BLI instruments. In a typical experiment, small animals (mice or rats) are placed into the “black box” of an IVIS Spectrum camera (PerkinElmer, USA) or similar instrument and an optical signal is detected using a “charged coupled device.” A larger non-rodent animal or human can't be placed in the IVIS camera. This represents another serious limitation for the use of this technology for drug development because a big fraction of in vivo tests are usually performed on non-rodent animal models. Animals must be anesthetized during the signal acquisition. This can drastically change their metabolism and affect multiple processes in the body. This is arguably the main reason why none of the current technologies have been adopted by pharmaceutical companies for the use in drug development research. The use of luciferase-expressing transgenic mice or transplanted cells together with the stationary BLI instruments can also be a rather expensive study. This issue can significantly restrict the use of the current BLI technologies by small companies and university laboratories.

To the best of our knowledge there are no patents or other publications describing the use of an isolated and/or substantially purified luciferase enzyme as an in vivo biosensor combined with a portable bioluminescence detector for the in vivo measurement of uncaged luciferin.

### BRIEF SUMMARY

In one aspect, a system comprising an implantable luciferase biosensor and a caged luciferin is provided. The implantable luciferase biosensor comprises luciferase. The system is a bioluminescence system for in vivo monitoring of a biological process or function in a subject, wherein when the caged luciferin is a substrate for the biological process or function, free or uncaged luciferin is released. The uncaged luciferin can then be a substrate for the luciferase such that luciferase activity in or near the implantable luciferase biosensor generates photons (light). The photons can then be detected in the live animal.

In some embodiments, the biological function or process is an activity of an enzyme. In some embodiments, the caged luciferin probe releases free luciferin upon activation of the enzyme. In other embodiments, the caged luciferin probe releases free luciferin upon inhibition of the enzyme.

In some embodiments, the biological function is intestinal absorption, wherein upon absorption of the caged luciferin probe in the intestine of the subject, free luciferin is released by the caged luciferin probe.

In some embodiments, the caged luciferin probe is not a substrate for the activity.

In some embodiments, the subject is a human, a non-human primate, or an animal. In other embodiments, the animal is a rat or mouse.

In some embodiments, the subject does not express luciferase. In other embodiments, the subject does not express endogenous luciferase. In still other embodiments, the subject does not express exogenous or recombinant luciferase.

In some embodiments, the implantable luciferase biosensor comprises a biocompatible matrix and the luciferase, wherein the luciferase is within or encased by the matrix. In other embodiments, the implantable luciferase biosensor comprises an encapsulation device (ED), wherein the luciferase is within or encased by the ED. In yet other embodiments, the ED does not comprise cells.

In some embodiments, the biocompatible matrix comprises a material selected from the group consisting of matrigel, one or more biocompatible polymers, a biocompatible hydrogel, peptide nanofibers and nanoparticles. In other embodiments, the matrix comprises matrigel.

In some embodiments, the matrix is semipermeable. In other embodiments, the matrix is permeable to uncaged luciferin. In still other embodiments, the matrix is not permeable to caged luciferin.

In some embodiments, the luciferase is an isolated luciferase. In other embodiments, the isolated luciferase was isolated from a cell expressing the luciferase prior to formulating the implantable luciferase biosensor.

In some embodiments, the luciferase present in the implantable luciferase biosensor is expressed by a cell which is present in the implantable luciferase biosensor. In other embodiments, the cell expresses a secreted form of the luciferase.

In some embodiments, the luciferase biosensor comprises a cell encapsulation device (CED), wherein the CED contains the cell expressing the luciferase.

In some embodiments, the luciferase is a firefly luciferase. In other embodiments, the luciferase is a red or green click beetle luciferase.

In some embodiments, the biosensor further comprises one or more components which facilitate luciferase activity. In other embodiments, the one or more components is selected from the group consisting of luciferase cofactors and luciferase activators. In still other embodiments, the one or more components is selected from the group consisting of Mg2+, ATP, ADP, adenylate kinase, CoA, dithiothreitol (DTT), inosine-5′-triphosphate, sodium tripolyphosphate, potassium pyrophosphate, and a combination thereof.

In some embodiments, the caged luciferin probe is selected from the group consisting of peroxy-caged luciferin, caspase substrate-caged luciferin, complementary caged luciferin precursors, organophosphine-caged luciferin, fatty acid-caged luciferin, beta-lactam caged luciferin, and galactoside-caged luciferin. In other embodiments, the caged luciferin probe is a substrate for a protease. In still other embodiments, the caged luciferin probe is a substrate for dipeptidyl peptidase IV or bacterial nitroreductase (NTR). In some embodiments, the caged luciferin probe is a prodrug.

In some embodiments, the caged luciferin probe releases free luciferin upon interaction of the caged luciferin probe with a 2-cyanobenzothiazole (CBT) compound. In other embodiments, the CBT compound comprises 6-amino-2-cyanobenzothiazole (ACBT). In still other embodiments, the CBT comprises 6-hydroxy-2-cyanobenzothiazole.

In some embodiments, the free luciferin is D-luciferin or D-aminoluciferin.

In some embodiments, the caged luciferin probe comprises luciferin or derivative thereof covalently bound to a caging moiety. In other embodiments, caged luciferin probe consists of the luciferin or derivative thereof covalently bound to a substrate moiety, wherein the substrate moiety is a substrate of the biological activity. In still other embodiments, the caging moiety comprises a dipeptide bond between two amino acids.

In some embodiments, the system further comprises a light detector. In other embodiments, the light detector is a portable light detector. In still other embodiments, the light detector is a hand-held light detector. In yet other embodiments, the light detector is an implantable light detector.

In some embodiments, the light detector is a stationary light detector. In other embodiments, the stationary light detector comprises a compartment into which the subject can be placed.

In some embodiments, the light detector is not a stationary light detector.

In some embodiments, the light detector can detect photon emission by the implantable luciferase biosensor. In other embodiments, the light detector can quantitatively measure photon emission by the implantable luciferase biosensor. In still other embodiments, the light detector can generate a signal which indicates the relative level of bioluminescence generated by a subject implanted with an implantable luciferase biosensor.

In some embodiments, the light detector detects ultraviolet (UV), visible and/or infra-red (IR). In some embodiments, the light detector is replaced with an instrument that can detect changes in temperature.

In some embodiments, the light detector is highly sensitive. In other embodiments, the detector has a low noise photodiode amplifier.

In one aspect, a method for monitoring a biological activity in an animal is provided. In some embodiments, the activity is enzyme activity. In still other embodiments, the biological activity cleaves a caged luciferin probe to release free luciferin. In other embodiments, the biological activity facilitates cleavage of the luciferin probe by an intermediate agent.

In some embodiments, the method comprises implanting into the animal an implantable luciferase biosensor, administering to the animal a caged luciferin probe, and detecting luminescence generated within the animal.

In some embodiments, the method comprising implanting the biosensor subcutaneously.

In some embodiments, the administering of the caged luciferin probe is by oral administration, subcutaneous injection, subcutaneous depot, or intravenous injection or infusion.

In some embodiments, the implantable luciferase biosensor is implanted prior to the administering the caged luciferin probe. In other embodiments, the implantable luciferase biosensor is implanted at the same time as the administering the caged luciferin probe. In yet other embodiments, the implantable luciferase biosensor is implanted after the administering the caged luciferin probe.

In some embodiments, the light detector is put into contact with the skin.

In some embodiments, the light detector is implanted together with the implantable luciferase biosensor.

In some embodiments, the detecting luminescence comprises using a portable light detector. In still other embodiments, the detecting luminescence comprises holding the portable light detector less than 1, 2, 3, 6 or 12 inches from the subject.

In some embodiments, the light detector is a stationary detector.

In some embodiments, the light detector comprises a compartment into which the subject can be place during detection of the light.

### BRIEF DESCRIPTION OF DRAWINGS

FIGS. 1A-1B illustrate a concept of bioluminescent imaging. FIG. 1A depicts oxidation of luciferin by luciferase resulting in photon emission. FIG. 1B illustrates a major principle of probe design-enzyme-mediated uncaging which serves as a readout of enzymatic activity.

FIG. 2 illustrates a system comprising implantation of a luciferase-containing moiety or device into a mice follow by administration of an activatable probe.

FIGS. 3A-3D provide data from a system used to detect a bioluminescent signal in animals injected with a luciferase matrigel plug and monitored with an IVIS Spectrum instrument. FIG. 3A: 100 mM luciferin; FIG. 3B: 10 mM luciferin; FIG. 3C: 1 mM luciferin;

FIG. 3D: linear correlation between luciferin concentration and signal strength.

FIGS. 4A-4D provide data from a system used to detect a bioluminescent signal in animals injected with a luciferase matrigel plug and monitored with a portable light detector. FIG. 4A: 100 mM luciferin; FIG. 4B: 10 mM luciferin; FIG. 4C: 1 mM luciferin; FIG. 4D: linear correlation between luciferin concentration and signal strength.

FIG. 5 illustrates a correlation between data obtained from a portable light detector and a non-portable light detector.

FIGS. 6A-6B provide data from an experiment in which an animal implanted with an implantable luciferase biosensor is treated with a caged luciferin probe for an enzyme in the absence (FIGS. 6A-6B, column 1) or presence (FIGS. 6A-6B, column 2) of an inhibitor of the enzyme. Bioluminescence was detected with a non-portable (FIG. 6A) or portable (FIG. 6B) imagine system.

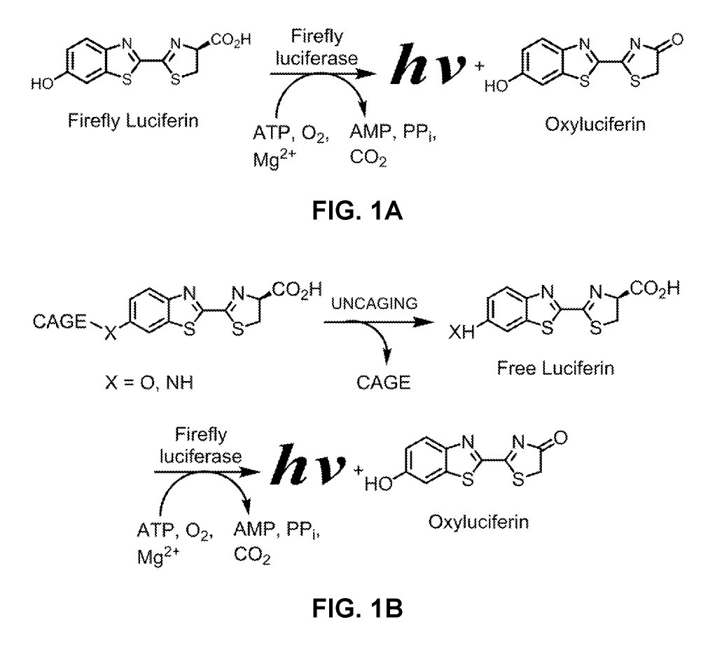

### DETAILED DESCRIPTION

### Definitions

“Luciferase,” as used herein refers to an enzyme that oxidizes a corresponding luciferin, thereby causing bioluminescence. Luciferase enzymes can be found in bacteria, fireflies, fish, squid, dinoflagellates, and other organisms capable of bioluminescence. Luciferase, as used herein, can include prokaryotic and eukaryotic luciferases, as well as variants possessing varied or altered optical properties, such as luciferases that generate luminescence at wavelengths in the electromagnetic spectrum.

“Luciferin,” as used herein refers to a substrate for a luciferase enzyme. Luciferins typically undergo an enzyme-catalyzed oxidation and the resulting excited state intermediate emits light (photons) upon decaying to its ground state.

“Caged luciferin” or “caged luciferin probe” as used herein are used interchangeably herein to refer to a composition comprising luciferin and a second moiety which is associated with the luciferin such that the luciferin is not accessible to the luciferase in the system. The caged luciferin is preferably a substrate of an enzyme of interest, although it is also contemplated that the caged luciferin can be the substrate of a second enzyme which is turn is activated or inhibited by a first enzyme or other molecule.

“Immobilize” as used herein refers to the ability of an element such as a matrigel plug to prevent release of a molecule such as luciferase from the element. In some embodiments, the element allows no more than 1% to 5% of the molecule to be released from the element.

“Subject,” “animal,” “host” and “individual,” as used herein are used interchangeably herein to a multicellular organism that is a member or members of any mammalian or non-mammalian species. Subjects thus include, without limitation, primate (including humans and non-human primates), rodents (e.g., mouse, rat), canine, feline, ungulate (e.g., equine, bovine, swine (e.g., pig)), avian, and other subjects. Humans are of particular interest in some embodiments. Non-human mammals having commercial importance (e.g., livestock and domesticated animals) are of particular interest in some embodiments. The term “multicellular organism” includes humans, non-human animals, and plants. Where a multicellular organism is an animal (humans and non-human animals), “multicellular organism” can be used interchangeably with “individual.”

For veterinary applications, a wide variety of subjects will be suitable, e.g., livestock such as cattle, sheep, goats, cows, swine, and the like; poultry such as chickens, ducks, geese, turkeys, and the like; and domesticated animals particularly pets such as dogs and cats. For diagnostic or research applications, a wide variety of mammals will be suitable subjects, including rodents (e.g., mice, rats, hamsters), rabbits, primates, and swine such as inbred pigs and the like. In some embodiments, a system includes a sample and a subject. The term “living host” refers to host or organisms noted above that are alive and are not dead. The term “living host” refers to the entire host or organism and not just a part excised (e.g., a liver or other organ) from the living host.

The term “generating an image” as used herein refers to acquiring a detectable signal generated from a luciferase light source according to the present disclosure and determining the location of the source in a cell or an animal or human tissue.

The term “portable light detector” or “hand-held light detector” as used herein refers to a light detector which can be carried by a single human for a time period (e.g., at least 5 minutes, 10 minutes, 30 minutes or 1 hour). A portable light detector is one that can be easily moved from one experimental subject to another by a human. The term “stationary light detector” as used herein refers to a light detector that is not moved from a location during or between experiments for which the light detector is used. The description may use both “photon” and “light” detection to describe the electromagnetic radiation emanating from and detected by a bioluminescence system. It is understood that electromagnetic radiation includes UV, IR and visible light and emission of electromagnetic radiation can cause temperature changes in the subject.

An in vivo Bioluminescence System

The present disclosure relates to compositions, systems and related methods that may be advantageously used for in vivo monitoring of biological processes in non-luciferase expressing subjects, including but not limited to small animals, large animals, humans and non-human primates. In some embodiments, the subjects do not need to be anesthetized during imaging. Specifically, the systems and compositions comprise an implantable luciferase biosensor and a caged luciferin.

The presently described systems allow real-time non-invasive evaluation of enzymatic activity and metabolic fluxes in non-luciferase expressing animals. In some embodiments, the subjects do not express endogenous luciferase. The subjects in which the systems may be used include both large and small animals as well as in humans. The systems can function as important tools for diagnostics and drug discovery.

The compositions and methods described herein are based on a highly sensitive imaging modality. Specifically, a better sensitivity is achieved in comparison to ones based on tissue and/or blood sampling. Another significant advantage of the presently described systems is the ability to obtain kinetic longitudinal readout of enzymatic activity, without any need of multiple tissue manipulation steps.

A further advantage of the systems and methods described herein is that the number of subjects can be significantly reduced because the technique is highly non-invasive and subjects can be repeatedly used for multiple assays. Moreover, the workload associated with tissue manipulation steps is considerably reduced. Each of these simplifications clearly has a positive impact on feasibility of such studies.

The presently described systems and methods allow for the study of metabolite absorption in both a real-time and a non-invasive manner, something that has not been achievable at the time of filing.

Specifically, the system comprises a biocompatible implantable luciferase biosensor and a caged luciferin is useful for in vivo monitoring, in whole animals or subjects, of biological processes such as enzyme function. The systems can also be used to monitor physiological processes or activities in the subjects such as intestinal absorption. In a preferred embodiment, the implantable biosensor is placed under the skin of the subject (subcutaneous implantation) and incorporates use of a light detector to produce a qualitative and/or quantitative readout of any free luciferin concentration near a luciferase. The readout is indicative of the biological processes or physiological activities in the subject being studied.

### The Implantable Luciferase Biosensor

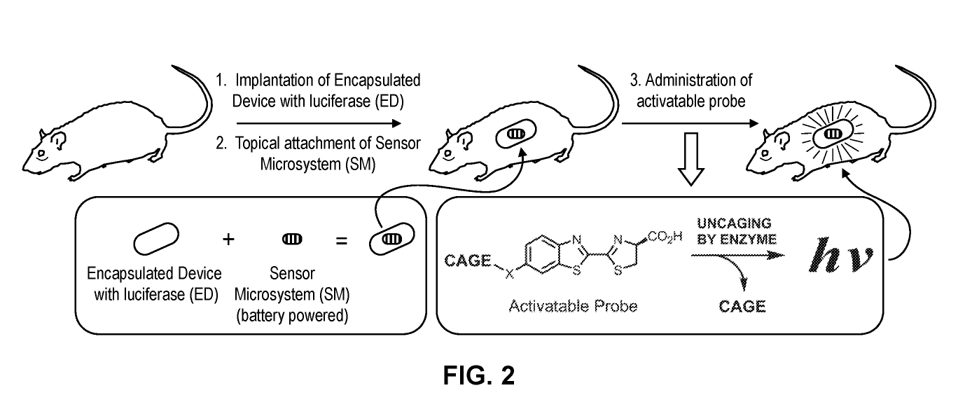

An example of biosensor system according to the present disclosure is illustrated in FIG. 2 and includes an implantable luciferase-containing biosensor and a caged luciferin. In this embodiment, the biosensor comprises an encapsulation device (ED) in which the luciferase are encased or contained. In some embodiments, the ED is a cell encapsulation device (CED) in which cells that express a luciferase are encased or contained. The cells may be engineered to harbor a luciferase expression vector wherein the luciferase-encoding gene comprises a nucleotide sequence encoding a signal sequence and the luciferase protein such that the expressed luciferase is secreted by the cells in the biosensor.

In preferred embodiments, the biosensor does not comprise cells that express luciferase. Instead, the luciferase present in the biosensor is one that was isolated and/or purified from a cell which expresses the luciferase. In this embodiment, the luciferase is mixed with a biocompatible matrix which is favorable to or compatible with luciferase activity. A biocompatible matrix may comprise, e.g., matrigel which can be described as a gelatinous protein mixture secreted by cells. Alternatively or additionally, the biocompatible matrix can comprise one or more extracellular matrices (containing laminin, collagen, proteoglycans, entactin/nidogen etc.), biocompatible polymers, a biocompatible hydrogel, Qgel, alginates or pectins, peptide nanofibers, or other nanoparticles.

The biocompatible matrix can be one which does or does not allow diffusion of the luciferase out of the matrix or biosensor. The biocompatible matrix can be semipermeable. For example, the biocompatible matrix can be permeable to uncaged or free luciferin. In some embodiments, the biocompatible matrix is not permeable to caged luciferin.

### The Luciferase

The luciferase included in the implantable luciferase biosensor can be from any species which naturally expresses luciferase. Examples of luciferase sources and corresponding substrates include but are not limited to firefly (Fluc/D-luciferin), green click beetle (CB green luciferase/D-luciferin), red click beetle (CB red luciferase/D-luciferin), Renilla reniformis (Rluc/Rluc8/Coelenterazine), Gaussia princeps (Glue luciferase/Coelenterazine), Aequorea victoria (Aequorin luciferase/Coelenterazine) Vargula hilgendorfii (Vluc luciferase), bacterial luciferase (LuxAB/Aliphatic aldehyde and reduced flavin mononucleotide phosphate). The firefly luciferase existing systems and methods are preferred for the present systems, compositions and methods.

### Caged Luciferin

The system further comprises a caged luciferin (alternatively referred to herein as a caged luciferin probe). Caged luciferin refers to a luciferin molecule that is associated with a moiety that prevents the luciferin from acting as a substrate for luciferase activity. The moiety can either prevent the luciferin from interacting with the luciferase in the biosensor by preventing any movement of the luciferin to the location of the luciferase and/or may modify the structure of the luciferin such that the luciferase cannot use the luciferin as a substrate even when the luciferin is in contact with the luciferase.

The term “caged” is utilized as an indication that a biologically or biochemically active species is trapped and masked inside a larger chemical “framework” and can be “released” upon, e.g., enzymatic cleavage of the caged probe or illumination, thus uncaging the active content. The term “caged” has become popular because it is brief and pictorial, rather than being strictly accurate (see Adams et al., Annu. Rev. Physiol. 55: 755-784, 1993).

The caged luciferin probe can be any molecule, compound or composition which is a substrate for an enzyme or other active biologic molecule of interest, and which is cleaved or altered to release free luciferin in the presence of the enzyme or other active biologic molecule of interest. In some embodiments, the caged luciferin probe does not release the free luciferin in the presence of the enzyme or other active biologic molecule when an inhibitor of the enzyme or other active biologic molecule of interest is near the caged luciferin probe.

In other embodiments, the caged luciferin probe releases free luciferin upon exposure to a biological process such as intestinal absorption. For example, upon absorption of the caged luciferin probe, free luciferin is able to enter the blood and encounter an implanted biosensor comprising luciferase.

In some embodiments, caged luciferin probe is injected in test subjects that were previously implanted with a luciferase biosensor. Alternatively, the caged luciferin probe is injected into test subjects prior to implantation of the luciferase biosensor. Free luciferin is then produced as a product of uncaging of the probe by a specific enzyme of interest or cellular internalization of metabolite. The amount of free luciferin produced is therefore proportional to the activity of the enzyme or uptake of metabolite and can be detected by luciferase sensor.

A variety of luciferin-based probes have been designed and described in research and patent literature. Examples of caged luciferin probes include peroxy-caged luciferin for monitoring oxidative stress (see, e.g., U.S. Pat. Appn. Pub. Nos. 2013/0315829 and 2015/0376680, U.S. Pat. No. 8,937,183, and PCT Pub. No. WO 2011/133800 A1); caspase substrate-caged luciferin for detection apoptosis and apoptosis-related diseases (see, e.g., U.S. Pat. No. 7,148,030, U.S. Pat. Appn. Pub. Nos. 2010/0303728 and 2009/0238766); complementary caged luciferin precursors for simultaneous monitoring oxidative stress and inflammatory processes (see, e.g., U.S. Pat. No. 9,173,966 and U.S. Pat. Appn. Pub. No. 2013/0287699 A1; organophosphine-caged luciferin for detection fluxes of low molecular weight (MW) metabolites, including glucose and lactate (see, e.g., PCT Pub. No. WO 2014/111906 A1); fatty acid-caged luciferin for detection of adipose tissue, fatty acid uptake rate, etc. (see, e.g., U.S. Pat. Appn. Pub. No. 2014/0199239); beta-lactam caged luciferin for evaluation of pathogen resistance to beta-lactam antibiotics (see, e.g., U.S. Pat. Appn. Pub. No. 2009/0246862); and galactoside-caged luciferin for investigation beta-galactosidase activity as well as cell to cell interaction (see, e.g., U.S. Pat. No. 7,582,417). The caged luciferin probe can be a substrate for bacterial nitroreducatases as described, for example, in Vorobyeva et al. (2015, PLOS ONE, 10:0131037). Alternatively, the caged luciferin probe is a substrate for a peptidase. For example, Dahan et al. (2014, Mol. Pharmaceutics, 11:4385-4394) describes a caged luciferin molecule as a prodrug which is a substrate for the protease dipeptidyl peptidase IV.

The presently described in vivo bioluminescence systems may also be used in conjunction with a “split luciferin reaction,” which is based on the fact that luciferins caged on the phenolic oxygen or aryl nitrogen do not lead to light production. Accordingly, in some embodiments, the caged luciferin releases an uncaged fragment or derivative which can interact with a 2-cyanobenzothiazole (CBT) compound (e.g., Hauser et al., 2016, J Org Chem., 12:2019-2025; Beilstein J. Org. Chem., 12:2019-2025; Godinat et al., 2013, ACS Chem. Biol., 8:987-999 and PCT Pub. No. WO 2014/057139). In some embodiments the CBT is 6-amino-2-cyanobenzothiazole (ACBT). In other embodiments, the CBT is 6-hydroxy-2-cyanobenzothiazole.

### The Light Detector

The system further comprises a light detector. The light emission from the luciferase sensor is detected by a sensitive light detector.

In some embodiments, the light detector is placed on top of the luciferase sensor, such as on the surface of the subject's skin near or at the location of implantation of the biosensor. Alternatively, the light detector can be implanted together with the implantable luciferase biosensor. The light emission is directly proportional to the total free luciferin concentration in the blood and, in turn, in interstitial fluid of the test subject.

In a preferred embodiment, the light detector is a portable light detector. In other words, the portable light detector can be easily carried between experimental sites by a single person. Moreover, the portable light detector can be easily manipulated to allow a user to position the detector in contact with the subject or within 1, 2, 3, 6, or 12 inches of the subject. The luminescence generated by the luciferase activity in the presence of uncaged (free) luciferin can be able to penetrate the skin of the subject so that the photons escape the subject and are detectable by the light detector.

It is understood by the ordinarily skilled artisan that any suitable photon detector may be used in the presently described systems. For example, photon detectors used in the system include but not limited to, photon detectors that detect visible light, ultraviolet (UV) light, infrared light and temperature. The detector is preferably located close to the sensor. The detector can be positioned in a location remote from the biosensor implanted in the animal as long as the detector can detect photons emitted by the system. Moreover, any suitable type of caged luciferin probe can be used. Several biosensors may also be simultaneously used. In some embodiments, different biosensors generate light having different ranges of wavelength.

### Light Detection and Detectors

Light generated per the disclosed compositions, methods and systems is luminescent. That is, the compositions and systems emit electromagnetic radiation in ultraviolet (UV), visible and/or infra-red (IR) spectra from atoms or molecules as a result of the transition of an electronically excited state to a lower energy state, usually the ground state.

An important aspect of the present disclosure is the selection of light-generating moieties that produce light capable of penetrating animal tissue such that it can be detected externally in a non-invasive manner. The ability of light to pass through a medium such as animal tissue (composed mostly of water) is determined primarily by the light's intensity and wavelength.

Luminescence generated within cells or animals implanted with the biosensors according to the present disclosure can be detected using a number of common imaging devices. The most common form of such devices detects emitted photons using a lens focused onto a cooled charge-coupled device (CCD) with or without pre-intensification via a photomultiplier tube (e.g. IVIS imaging systems, by Perkin Elmer, MA, USA; and Photon Imager system, by Biospace Laboratories, Paris, France).

In preferred embodiments, the light detector is a portable and/or hand-held light detector. Any, sufficiently sensitive detector of light, including but not limited to amplified and not amplified photodiodes, cmos sensors, avalanche photodiodes, photomultiplier tubes, single photone avalanche diodes, transition edge sensors, superconducting nanowire single photon detectors, visible light photon counters, and other detectors or detector arrays.

### Methods of Use

The systems and compositions described herein are useful in monitoring biological, e.g., enzymatic, activities within a living subject. Accordingly, the present disclosure provides methods for administering to the living subject an implantable luciferase biosensor and a caged luciferin probe. A subject is implanted with an implantable luciferase biosensor as described here. The implant can be at any location where the biological activity is expected to occur. In some embodiments, an implantable light detector can be implanted near the biosensor implant.

Before, concurrent with, or after implantation of the biosensor, a caged luciferin is administered to the subject. A composition comprising the caged luciferin can be administered orally, parenterally, by inhalation spray, topically, rectally, nasally, buccally, vaginally or via an implanted reservoir. The caged luciferin can be administered at a location near or distant from the biosensor implant.

The caged luciferin composition can contain any conventional non-toxic pharmaceutically-acceptable carriers, adjuvants or vehicles. The term parenteral as used herein includes intraperitoneal, subcutaneous, intracutaneous, intravenous, intramuscular, intra-articular, intrasynovial, intrasternal, intrathecal, intraocular, intraorbital, intralesional and intracranial injection or infusion techniques. Preferably, the route of administration of the composition is intraperitoneal, intravenous or intramuscular administration (most preferably intraperitoneal).

After administration of both the biosensor and the caged luciferin, a light detector is used to detect and/or measure generation of release of light (photons) from the animal. Preferably, the light detector is positioned close to the biosensor implant in the animal. Light generation by the biosensor can be detected by either a portable or stationary light detector, although is portable detector is usually preferred. The light detector can either comprise an analyzer component (e.g., microprocessor) and output device to provide a visible detection signal or quantitative measure of the generated light, or can be connected to an analyzer component and output device.

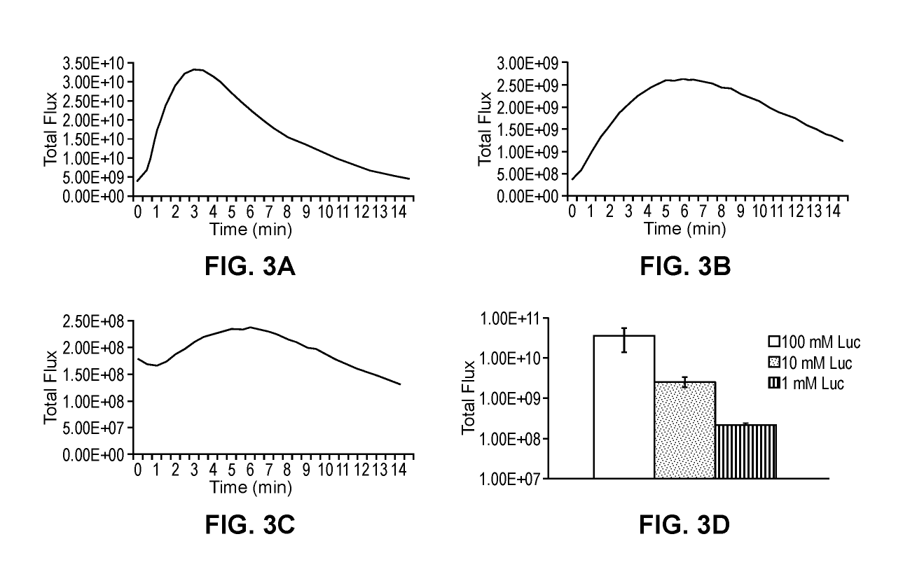

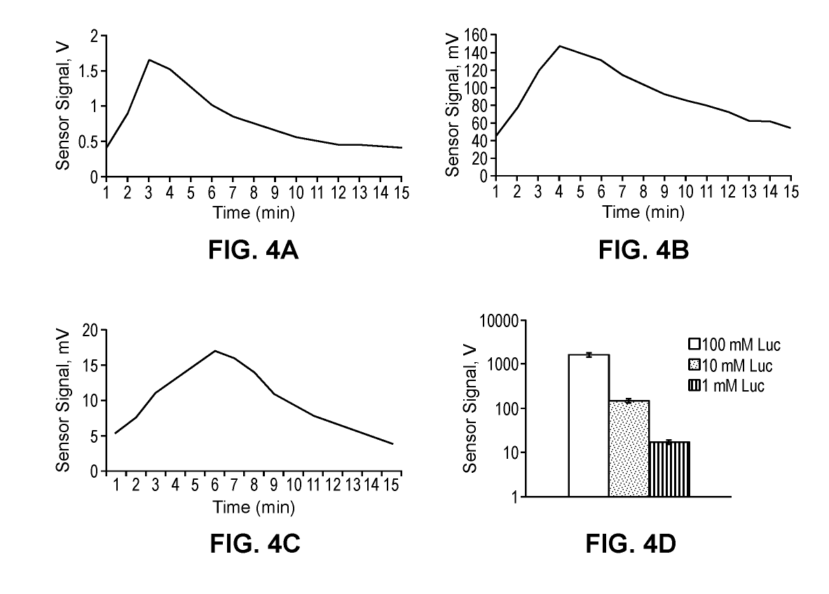

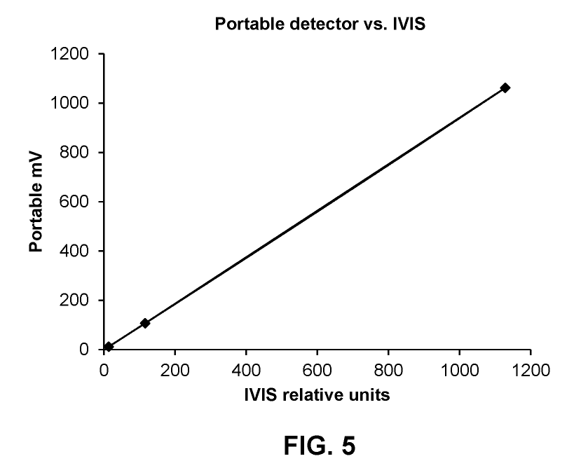

Examples 1-3 below describe use of bioluminescence systems with light detector devices to demonstrate the effectiveness of the systems to monitor biological processes in whole animals. As described in Examples 1 and 2, an implantable luciferase biosensor was formulated which comprises free luciferase enzyme in a matrigel matrix. Free luciferin was then administered to the mice at concentrations ranging from 1 mM to 100 mM. Light generation was then measured by using a stationary light detector (Example 1 and FIGS. 3A to 3D) or a portable light detector (Example 2 and FIGS. 4A-4D). As summarized by the graph shown in FIG. 5, there is a very high correlation between the data obtained with a portable light detector and data obtained using a stationary light detector. These experiments show that portable light detectors are highly effective in detecting and measuring luminescence generated in vivo in living subjects using the bioluminescence systems described herein.

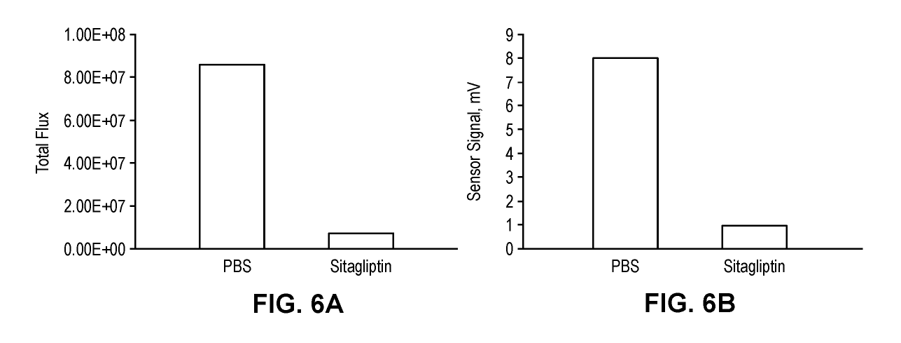

Example 3 describes an experiment performed to show the efficacy of the bioluminescence systems for monitoring in vivo biological (enzyme) activity in living subjects using a caged luciferin probe which is a substrate for an endogenously expressed enzyme. In Example 3, the caged luciferin probe is a substrate for the protease dipeptidyl peptidase IV (DPP IV). When active DPP IV is expressed by the subject and present near the caged luciferin probe, the DPP IV activity cleaves the caged luciferin to release free luciferin. In Example 3, subjects were implanted with an implantable luciferase biosensor comprising substantially pure luciferase and then administered the caged luciferin probe. Half of the subjects were also administered an inhibitor of DPP IV while the other half of the subjects were administered phosphate buffered saline (PBS) as a negative control. As shown in FIGS. 6A-6B, administration of the inhibitor (column 2) resulted in a significant decrease in light generation as compared to light generated in control subjects treated with PBS. The quality of light detection was equivalent for the stationary light detector (FIG. 6A) and the portable light detector (FIG. 6B).

### Bioluminescence System Kits

Also provided herein is a kit comprising components that make up a kit for use in in vivo monitoring of molecular and metabolic events in animal models.

### EXAMPLES

### Example 1

### In Vivo Detection of Luciferase Activity Using an IVIS Spectrum Instrument

Studies were performed to demonstrate the ability to detect a bioluminescent signal from luciferase matrigel plugs using the noninvasive IVIS Spectrum In Vivo imaging system (Perkin Elmer). Plugs were formulated to contain 50 μl matrigel (Sigma-Aldrich, St. Louis, Mo., Cat. No E6909), 1 μg of free luciferase enzyme (Sigma-Aldrich, St. Louis, Mo., Cat. No SRE0045; 1×10¹¹ units per mg protein), 5 mM Mg²⁺ ions and 2 mM ATP (100 μL total volume in PBS). The plugs (100 μL per injection) were injected under the skin at the dorsal side of nude mice. After injection of the plugs, different luciferin concentrations (100 mM, 10 mM or 1 mM in 100 μL of PBS) were administered intraperitonealy to the mice (3 mice per group). The animals were placed into the IVIS Spectrum instrument immediately after administration of the luciferin and the BLI signal was monitored every minute for at least 15 minutes (see FIGS. 3A (100 mM luciferin), 3B (10 mM luciferin) and 3C (1 mM luciferin)). Linear correlation between luciferin concentration and signal strength was observed (FIG. 3D).

### Example 2

### In Vivo Detection of Luciferase Activity Using a Portable Light Detector

Studies were also performed to demonstrate the ability of a bioluminescence sensor microsystem to detect luciferase activity in vivo. Again, plugs formulated to contain 50 μl matrigel (Sigma-Aldrich, St. Louis, Mo., Cat. No E6909), 1 μg of free luciferase enzyme (Sigma-Aldrich, St. Louis, Mo., Cat. No SRE0045; 1×10¹¹ units per mg protein), 5 mM Mg²⁺ ions and 2 mM ATP (100 μL total volume in PBS) were injected under the skin at the dosal side of nude mice. After injection of the plugs, different luciferin concentrations (100 mM, 10 mM or 1 mM in 100 μL of PBS) were administered to the mice (at least 3 mice per group).

Immediately after injection of the luciferin, a portable BLI sensor based on amplified photodiode was placed on top of the plug and the BLI signal was monitored every minute in the dark chamber for at least 15 minutes (see FIGS. 4A (100 mM luciferin), 4B (10 mM luciferin) and 4C (1 mM luciferin)). Linear correlation between luciferin concentration and the peak of BLI signal was observed (see FIG. 4D).

The data from Examples 1 and 2 were analyzed to determine a positive correlation between detection of luciferase activity using the IVIS Spectrum instrument and the Portable Light Detector (see FIG. 5).

### Example 3

### Targeted Assay of Dipeptidyl Peptidase 4 In Vivo

A study was performed to do a direct comparison of IVIS Spectrum and sensor microsystem imaging of a dipeptidyl peptidase-4 (DPP4) caged luciferin probe. Wild type nude mice were implanted with a luciferase plug as described in Example 1 and divided into 2 groups, each group containing 3 mice. One group of mice was injected with sitagliptin, a well characterized inhibitor of DPP4 while the other was injected with PBS control. Caged luciferin based probe for DPP4 enzyme was then injected into both groups of mice and the signal was recoded using IVIS system (FIG. 6A) or sensor microsystem (FIG. 6B). Both imaging systems detected approximately 10 times lower bioluminescent signal from the inhibitor treated animals (FIGS. 6A and 6B, bar 2) as compared to animals treated with PBS (FIGS. 6A and 6B, bar 1).

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

What is claimed is:

1. A bioluminescence system comprising:
(a) an implantable luciferase biosensor capable of releasing photons generated by the reaction between a luciferase and an uncaged luciferin; and
(b) a caged luciferin probe comprising a luciferin;
wherein the caged luciferin probe can be modified upon exposure to a biological activity present in a subject to generate the uncaged luciferin.

2. The system of claim 1, further comprising: (c) a light detector which can detect and/or measure photon emission by the implantable luciferase biosensor.

3. The system of claim 2, wherein the light detector is a portable light detector.

4. The system of claim 1, wherein the implantable biosensor comprises the luciferase and wherein the luciferase was isolated from a cell that expressed the luciferase.

5. The system of claim 1, wherein the implantable biosensor comprises a cell which expresses the luciferase.

6. The system of claim 1, wherein the implantable biosensor comprises a biocompatible matrix capable of immobilizing the luciferase.

7. The system of claim 6, wherein the biocompatible matrix material is selected from the group consisting of matrigel, one or more biocompatible polymers, a biocompatible hydrogel, peptide nanofibers and nanoparticles.

8. The system of claim 1, wherein the implantable biosensor comprises luciferase-functionalized nanoparticles.

9. The system of claim 1, wherein the implantable biosensor comprises a semi-permeable matrix.

10. The system of claim 9, wherein the semi-permeable matrix is permeable to luciferin.

11. The system of claim 1, wherein the implantable biosensor further comprises one or more compounds required for luciferase activity.

12. The system of claim 11, wherein compounds favorable for luciferase activity are selected from the group consisting of Mg²⁺, ATP, ADP+ adenylate kinase, CoA, dithiothreitol, inosine 5′-triphosphate, sodium tripolyphosphate, and potassium pyrophosphate.

13. The system of claim 1, wherein said implantable biosensor comprises a cell encapsulation device with luciferase-expressing cells.

14. The system of claim 1, wherein the caged luciferin probe consists of the luciferin or a derivative thereof covalently bound to a substrate moiety.

15. The system of claim 1, wherein the caged luciferin or derivative thereof specifically binds a targeted intracellular compound, metabolite or enzyme and wherein binding to the targeted intracellular compound, metabolite or enzyme results in cleavage of the caged luciferin or fragment thereof to produce an active uncaged luciferin or fragment thereof.

16. The system of claim 15, wherein the uncaged luciferin derivative is able to form free luciferin upon in vivo interaction with a 2-cyanobenzothiazole (CBT) compound.

17. A method for in vivo monitoring of a biological process in a subject, comprising:
(a) implanting an implantable luciferase biosensor into the subject, wherein the biosensor comprises a luciferase and wherein the biosensor releases photons generated by the reaction between the luciferase and an uncaged luciferin;
(c) administering a caged luciferin probe into the subject; and
(d) measuring photon emission from the biosensor with a light detector;
wherein the caged luciferin is a substrate for a biological process and wherein the caged luciferin releases the uncaged luciferin when the biological process is active.

18. The method of claim 17, further comprising positioning a light detector topically, close to, or at a distance from the biosensor implanted in the subject, and measuring photon emission from the biosensor with the light detector.

19. The method of claim 18, further comprising evaluating the activity of the biological process based on the light detector measurements of photon emission.

20. A kit comprising:
(a) an implantable luciferase biosensor, wherein the biosensor comprises a luciferase; and
(b) a caged luciferin;
wherein the caged luciferin can release an uncaged luciferin in the presence of a functional activity in vivo, and wherein the biosensor is capable of releasing photons generated by a reaction between the luciferase and the uncaged luciferin.
